(19) United States
(12) Patent Application Publication
(10) Pub. No.: US 2026/0295125 A1
(43) Pub. Date: Oct. 1, 2026

(54) Implantable Composition For Use In Cartilage Defects Repair In Post-Surgery Rehabilitation

(71) Applicants: FONDAZIONERI, MED, Palmero (IT); THE CHILDREN'S HOSPITAL OF PHILADELPHIA, Philadelphia, PA (US)

(72) Inventors: Roberto DI GESU', Petralia Sottana (IT); Riccardo GOTTARDI, Philadelphia, PA (US)

(21) Appl. No.: 19/490,172

(22) PCT Filed: Jun. 8, 2023
(86) PCT No.: PCT/EP2023/065364

§ 371 Date: Dec. 4, 2025

(51) Int. Cl. A61L 27/44; A61L 27/04; A61L 27/06; A61L 27/22; A61L 27/38; A61L 27/52; A61L 27/54

(52) U.S. Cl. CPC A61L 27/446; A61L 27/042; A61L 27/06; A61L 27/222; A61L 27/3817; A61L 27/3834; A61L 27/52; A61L 27/54; A61L 2400/12

(57) ABSTRACT

The invention relates to an implantable composition, comprising, as main components, a piezoelectric polymer loaded with magnetic micro-or nanoparticles and cellularized with stem cells, chondrocyte progenitors, or adult chondrocytes; the composition is useful in the post-surgery regenerative rehabilitation as part of the treatment of articular diseases caused by chondral defects (e.g. osteoarthritis).

### FIELD OF THE INVENTION

The invention relates to a composition comprising, as main components, a piezoelectric polymer loaded with magnetic micro-or nanoparticles and cellularized with stem cells, chondrocyte progenitors, or adult chondrocytes; the composition is useful in the post-surgery regenerative rehabilitation as part of the treatment of articular diseases caused by chondral defects (e.g. osteoarthritis).

The invention was first disclosed at the 13^{th} Annual The Orthobiologic Institute (TOBI) Conference organized by The Orthobiologic Institute (TOBI), held Jun. 09-11, 2022, at The Diplomat Beach Resort Hollywood, Curio Collection by Hilton, Hollywood, Florida, United States of America.

### STATE OF THE ART

Musculo-skeletal diseases secondary to chondral-defects are disabling conditions that represents an extensive and increasing health burden with remarkable implications both for patients and for health-care systems and related costs. Among chondral defects-related pathologies, osteoarthritis is one of the most representative; due to the relevance and incidence of osteoarthritis, in the description that follows reference will mainly be made to this condition, but the invention may find general applicability in all diseases connected to chondral defects.

Several epidemiologic studies underline that the pathology is becoming more relevant year after year, with a high correlation with the ageing and increasing obesity in the global population. Such studies estimate that about 250 million people are currently affected by symptomatic activity-limiting osteoarthritis. To date no effective therapies have been developed, and the only resolutive treatment is the total joint replacement (TJR) surgery. However, several observational studies report that failure rate of TJR is still high in younger and more active recipients, those with comorbidities, and those who had surgery in low-volume center. In particular, it is worthy to be underlined that up to 20% of prosthesis need to be revised within 20 years.

More advanced and promising approaches are based on reparative cartilage techniques (RCT) such as microfracture (MF), autologous chondrocytes implantation (ACI), and matrix-assisted autologous chondrocyte implantation (MACI). The rationale is to induce a pro-regenerative response in situ stimulating the formation of new functional tissue to repair the cartilage defects. Unfortunately, the success rate of these innovative procedures is still too low. In fact, the main limitation of RCTs is the biologically inert nature of the matrix used as cells support. Several attempts have been tried to overcome that limitation, leading to focusing on the development of a pro-regenerative rehabilitation protocol relying on the pro-regenerative response of chondrocytes after well-defined mechanical stimulation. Unfortunately, the rehabilitation is not fully efficient due to the lack in technologies able to make effective mechanical stimuli at cellular level, inducing highly variable and inadequate positive effects in vivo.

In fact, regardless the reparative techniques adopted, a long and challenging post-surgery rehabilitation is needed to restore the pre-surgery overall mobility. In addition, each patient represents a unique case requesting a personalized rehabilitation protocol developed ad hoc. Such wide heterogeneity leads to a dilating of the recovery time, which may take up to 25 months.

Due to these complications, RCTs approaches still suffer high percentage of unsuccess that drastically limits the post-surgery outcomes, especially in young people.

The treatment of chondral defects-related pathologies is still an open question waiting for a clinical response.

To date, the gap in technology is the development of a matrix able actively support cells after the implantation, unify all methods already developed to express their full potential.

It is an object of the present invention to biofabricate a biocompatible material working as an active support in the RCTs surgery. Such kind of matrix is able to promote the pro-regenerative potential of cells through the simultaneous delivery of physical pro-regenerative cues in situ. Remarkably, the matrix will make it possible to perform a personalized pro-regenerative regimen at cellular level, with a cost-effective and minimally invasive approach.

The matrix described in this patent is ultimately intended for the use in post-surgery pro-regenerative rehabilitation as part of the treatment of the diseases etiologically linked to chondral defects (e.g. osteoarthritis).

### SUMMARY OF INVENTION

This and other objects are achieved with the present invention that, in a first aspect thereof, relates to a biocompatible composition for use in implants in an area of the body subjected to surgery for the treatment of pathologies characterized from chondral defects, comprising:
a) a matrix comprising a biocompatible polymer that is intrinsically piezoelectric, or a biocompatible polymer non-intrinsically piezoelectric loaded with an inorganic or organic filler imparting piezoelectric properties to the non-intrinsically piezoelectric polymer;
b) between 1×10⁵-1×10⁹ nanoparticles/ml of a magnetic material; and
c) between 1×10⁵ and 1×10⁸ cells chosen from the group consisting of mesenchymal stem cells, adult chondrocytes, and chondrocyte progenitors per milliliter of composition.

In a second aspect the present invention describes a method for the treatment of an articular disease in a subject in need thereof, said method comprising the step of using the biocompatible composition as herein described.

### BRIEF DESCRIPTION OF THE DRAWINGS

FIG. 1 shows the electric cues generated in a material of the invention upon compressive stress, without a pre-load (FIG. 1a) and with a pre-load of 5 N (FIG. 1b);

FIG. 2 shows the results of MRI imaging on one scaffold of the invention, respectively in a coronal view (FIG. 2a) and in sagittal view (FIG. 2b);

FIG. 3 shows X-ray measurements obtained on one scaffold of the invention, respectively before magnetic excitation (FIG. 3a) and after magnetic excitation (FIG. 3b)

### DETAILED DESCRIPTION OF THE INVENTION

In the rest of the description, the following terms and abbreviations are used, with the meanings reported below:
“NPs” stands for nanoparticles;
“ES” stands for electrospun
“MF” stands for microfracture
“MACI” stands for Matrix-assisted Autologous Chondrocytes Implantation
“PBS” stands for Phosphate-buffered Saline
“GeIMA” stands for Gelatin methacrylate
“hACs” stands for human articular chondrocytes
unless indicated otherwise, the percentages of components described below are weight/volume (w/v) percentages, indicating the grams of a component dissolved (or dispersed) in 100 ml of the composition;
unless indicated otherwise, the cell density is described as cell/ml, indicating the number of cells dispersed in 1 ml of suspending medium;

The inventors have found that a piezoelectric biocompatible matrix loaded with magnetic micro- or NPs and cellularized with chondrocyte progenitors also referred to as chondrocyte progenitor cells (e.g., perichondroblasts, synoviocytes, . . . ) , mesenchymal stem cells, or adult chondrocytes, implanted by MF/MACI in the site of a surgery intervention, is able to maximize the effects of post-surgery regenerative rehabilitation is subjected to magnetic irradiation, in particular in the treatment of osteoarthritis. Magnetic nanoparticles embedded in the polymeric matrix vibrate in response to a pulsatile external magnetic field and trigger a controlled deformation of the matrix. The piezoelectric polymer reacts to the deformation by generating an electrical discharge promoting type II collagen production and the progenitor cells differentiation toward chondrocyte-like phenotype (direct effect). Moreover, the cyclic deformation of the matrix, reproduces therapeutic shear/compressive deformation induced by the regenerative rehabilitative therapy (indirect effect). This approach allows for a strong in situ chondroinduction through a personalized and non-invasive approach.

The first component of the composition of the invention is or comprises a biocompatible polymer.

The polymer may be intrinsically piezoelectric, such as polyvinylidene difluoride (known in the field of polymers as PVDF), poly(vinylidene fluoride-trifluorethylene) (P(VDF-TrFE)), poly(L-lactic acid) (PLLA), poly(3-hydroxybutyrate-co-3-hydroxyvalerate) (PHBV), cellulose, collagen, polyacrylonitrile (PAN), and nylon-11, or mixtures thereof. These polymers may be used dissolved or suspended in suitable solvents, for instance water, PBS at pH 7.4, culture media (cellulose, collagen), tetrahydrofuran, diethyl ether (in particular when the polymer is PLLA), dimethylformamide, exafluoroisopropanol, methyl ethyl ketone (especially in combination with P(VDF-TrFE), in particular in the form of ES fibers), or mixtures thereof; the amount of polymer is typically between 10 and 20% w/v.

Alternatively, the biocompatible polymer may not be piezoelectric in itself, in which case it is loaded with micro-or nanoparticles of an inorganic filler. In this case, the polymer may be any, and preferred ones are polymers of natural origin, such as animal gelatins. These polymers may be employed in the form of a hydrogel, that is a polymer phase swelled by and incorporating water, PBS or culture media. When the matrix is a hydrogel, typically the amount of polymer ranges between about 2 and 30% w/v of the overall composition; a typical water-based phase for the preparation of hydrogels is a phosphate buffer with pH between 6 and 10. The non-piezoelectic polymers must be loaded with one or more inorganic fillers having piezoelectric properties; examples of these materials are barium titanate (BaTiO₃), lead zirconate titanate (Pb[ZrₓTi₁₋ₓ]O₃, 0≤x≤1), zinc stannate (ZnSnO₃), lithium niobate (LiNbO₃) and sodium niobate (NaNbO₃). These inorganic compounds are in form of NPs, that is, particles with diameter (or maximum axis) between 1 nm and 100 nm or in the form of microparticles with a diameter or maximum axis between 101 nm and 100 μm. The inorganic filler may be present in the composition in amounts between 4 and 40% w/v.

The second component of the composition of the invention is a magnetic material, in its turn in form of NPs and dispersed in the matrix.

Although many classes of magnetic materials are known (for instance, composite ceramic magnetic materials, single-molecule magnets or SMMs or the organic magnet of composition V(TCNE)₂. ½CH₂Cl₂, wherein TCNE stands for tetracyanoethylene), preferred for the objects of the present invention are magnetic metals, metal oxides or metal alloys. Examples of magnetic metals are iron, nickel and cobalt; examples of oxides are the oxides of the metals above, such as ferric oxide (Fe₂O₃), magnetite (Fe₃O₄) or mixed oxides including these metals; examples of magnetic alloys are steel, the alloys known as “AlNiCo” (comprising iron, nickel, cobalt and aluminum as main components), “TiCoNiAl” (comprising iron, nickel, cobalt, aluminum and titanium as main components), or the alloys based on rare-earth elements such as samarium-cobalt and neodymium-iron-boron magnets. For the objects of the invention, it is possible to use a mixture of NPs of different magnetic materials.

The magnetic NPs may be present in the composition in amounts between 1×10⁵ and 1×10⁹ NPs/ml.

Finally, the composition of the invention comprises between 1×10⁵ and 1×10⁸ cells chosen from the group consisting of mesenchymal stem cells, chondrocyte progenitors, or adult chondrocytes per milliliter. In a preferred aspect, the invention comprises between 2×10⁶ and 2×10⁷ cells chosen from the group consisting of mesenchymal stem cells, adult chondrocytes and chondrocyte progenitors cells per milliliter.

Further to the necessary components described above, the composition of the invention may comprise one or more additional components, such as antioxidants (e.g., ascorbic acid), preservatives (e.g., ethyl alcohol, benzyl alcohol, sodium benzoate, . . . ), or bioactive agents such as growth factors, drugs, antibiotics, antivirals, enzymes, and vitamins.

In a second aspect the present invention describes a method for the treatment of an articular disease in a subject in need thereof, said method comprising the step of using the biocompatible composition as herein described.

The patient or subject in need of being treated with the method for the treatment of an articular disease may be suffering from a chondral defect or from diseases etiologically linked to chondral defects, in particular the chondral defect may be osteoarthritis.

The invention will be further illustrated by the following examples.

### Example 1: Gelatin Based Hydrogel Doped With BaTiO₃ as Piezoelectric Inducer

This example refers to the fabrication of a scaffold based on gelatin from porcine skin enriched with a piezoelectric filler made of BaTiO₃ formulated as nanopowder. With this aim, a 5% w/v gelatin solution was prepared dissolving 500 mg of gelatin (Sigma-Aldrich, USA) with 10 ml of a 0.15% w/v genipin (Sigma-Aldrich, USA) solution in phosphate buffer saline (PBS) 1 M at pH 7.4 under magnetic stirring (600 rpm) at 60° C. 40% w/v of BaTiO₃ nanopowder was then gradually added to the mixture, allowing for a homogenous dispersion (the remainder part of the composition to 100% being represented by the PBS solution). Genipin acts a crosslinking agent, avoiding the dissolution of the gelatin scaffold after soaking in water, PBS, or culture medium. The magnetic stirring was stopped at complete solubilization, then, the gelatin/genipin solution was allowed to crosslink maintaining the solution at 60° C. The end point of the reaction was established following the color of the solution, which turns from a pale yellow to a mid-to-dark blue when carboxylic groups of genipin react with the amine groups of gelatin. In this specific case the solution was maintained at 60° C. for 15 minutes after the early appearing of a light blue color. Several discoidal scaffolds (∅=2.5 cm, h=0.5 cm) were fabricated.

Example 2: Measurement of the Electric Field Generated From the Gelatin/Genipin/BaTIO₃ Scaffold After Controlled Deformation

The main aim of this Example was to test the induced piezoelectricity properties on the scaffold produced in Example 1. One of the scaffolds produced in Example 1 was subjected to controlled deformation using an electromechanical testing system (Instron 5943, load measurement accuracy: +/−0.5% of reading, up to 2.5 kHz data acquisition rate option simultaneous on load, extension, and strain channels, speed range of 0.05-2500 mm/min (0.002-100 in/min), 1 kN (225 lbf) capacity, 1123 mm (44.2 in) vertical test space, INSTRON-USA). The produced electric field was measured using a digital multimeter GBC KDM-120 (GBC, Australia). Specifically, a 10% deformation was induced on the scaffold above described, reading out an electric field of 3 mV.

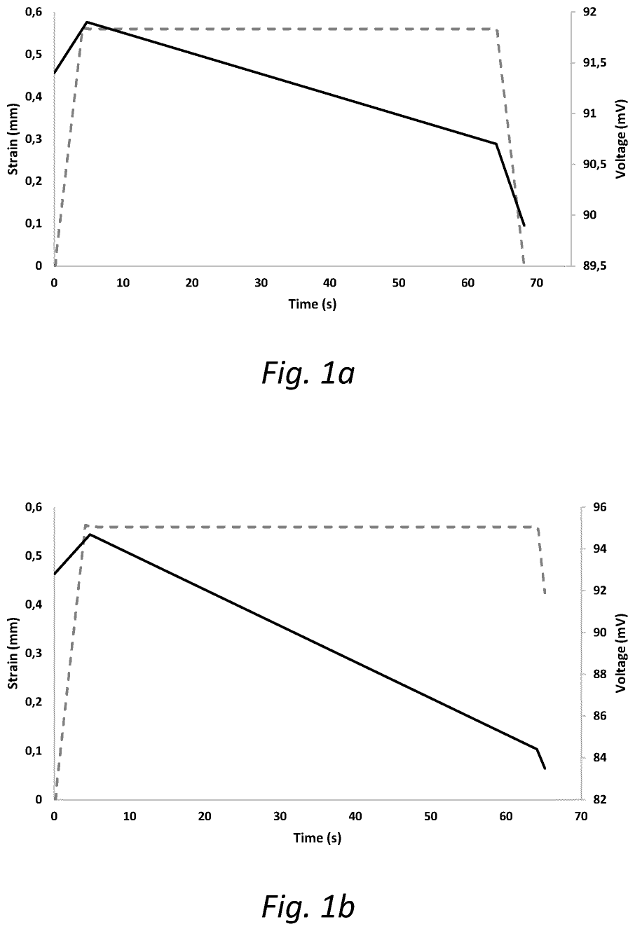

The results of this test are reproduced graphically in FIGS. 1a and 1b. In these figures, the dashed line refers to the applied strain, and the solid line to the generated voltage. In particular, FIG. 1a shows the voltage generated upon controlled compression cycles using a strain control approach. This kind of deformation generated a 2 mV variation of voltage (FIG. 1a).

FIG. 1b shows the variation of voltage when the same deformation was applied after a 5 N pre-load; in this case the difference of voltage increased up to 10 mV.

### Example 3: Gelatin Based Hydrogel Enriched With Ferromagnetic Microparticles to be Responsive to External Magnetic Fields

A hydrogel composed of gelatin from porcine skin crosslinked with genipin was produced using the methodic described in Example 1. Briefly, a 5% w/v gelatin solution was prepared dissolving 500 mg of gelatin (Sigma-Aldrich, USA) with 10 ml of a 0.15% w/v genipin (Sigma-Aldrich, USA) solution in phosphate buffer saline (PBS) 1M at pH 7.4 under magnetic stirring (600 rpm) at 60° C. When the mixture polymer/genipin was completely dissolved, a suspension of magnetic microparticles Dynabeads™ M-280 Streptavidin (Thermofisher, USA) was added under magnetic stirring. Specifically, 500 μl/ml of the microparticles suspension from a stock at 6.7×10⁸ beads/ml were used.

When a homogeneous suspension was obtained, the magnetic stirring was stopped, and the gelatin/genipin solution was allowed to crosslink maintaining the solution at 60° C. The solution was maintained at 60° C. for 15 minutes after the early appearing of a light blue color. Several disc-shaped scaffolds (∅=2.5 cm, h=0.5 cm) were fabricated.

### Example 4: Evaluation of the Deformation of a Gelatin-Based Hydrogel Enriched With Ferromagnetic Microparticles After Application of Controlled External Magnetic Fields

To evaluate the responsiveness of the scaffolds produced in Example 3, the hydrogel composed of gelatin/genipin enriched with magnetic microparticles was subjected to controlled external fields using a 0.3 T magnetic resonance imaging (MRI) apparatus. Specifically, each scaffold was subjected to cyclic magnetic fields, and the diameter was measured both in coronal and in sagittal view.

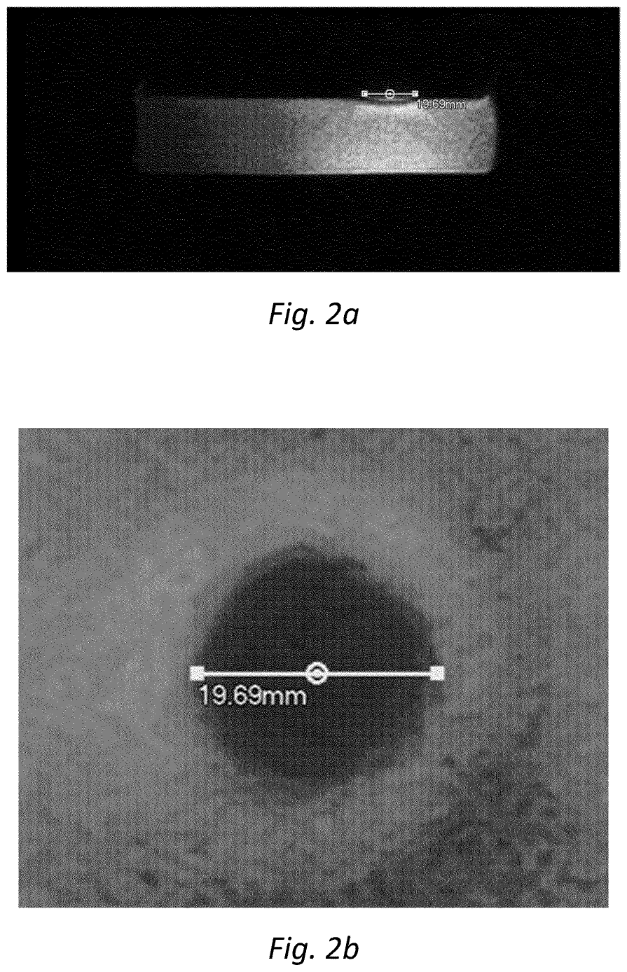

FIG. 2 shows the results of MRI imaging on one scaffold of the invention: in FIG. 2a the scaffold is shown in a coronal view, in FIG. 2b in sagittal view.

To precisely quantify the scaffold deformation, the diameter of each hydrogel immediately before and after the MRI cycle via X-ray analyses was measured.

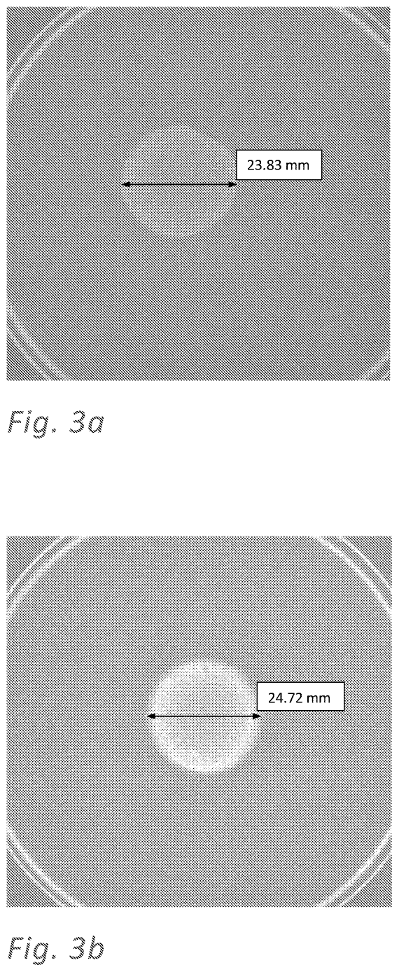

FIG. 3 reproduces the X-ray measurements obtained on one of the produced scaffolds. FIG. 3a shows the diameter of the scaffold (in a Petri dish), of 23.83 mm before excitation; FIG. 3b shows the diameter of the scaffold after excitation, resulting in this case 24.72 mm.

### Example 5: Cellularization of a Crosslinked Gelatin-Based Scaffold With Human Articular Chondrocytes (hACs)

Human chondrocytes (hACs) were harvested from healthy femoral condyles and tibial plateau cartilage obtained from donors subjected to total knee replacement (TKR) prosthetic surgery.

Specifically, tissues were harvested from 3 female donors (64, 71, and 82 years old), selected on the basis of definite inclusion criteria (unicompartimental osteoarthritis, no previous knee surgery, no relevant comorbidities). Surgery waste was carefully washed with sterile PBS and placed in a sterile plate containing dissection medium (Diss-M: DMEM high glucose—Gibco USA, Penicillin/Streptomycin/Amphotericin 1% v/v-Gibco USA, Fetal Bovine Serum 10% v/v—Gibco USA). Hence, healthy cartilage was carefully removed using a sterile scalpel avoiding to harvest the underlying subchondral bone, moved to a new sterile plate containing Diss-M, then minced in 1 cm² pieces. Subsequently, minced cartilage was collected into a sterile 500 ml, conical tube and incubated with a 5 mg/ml solution of Collagenase A (Worthington, UK) in Diss-M at 37° C. and mechanical agitation (250 RPM) overnight. Finally, cells were recovered, filtered through a 40 μm cell strainer, counted, and plated at 7.0×10³ cell/cm² into a tissue culture treated T75 flask. Chondrocytes were allowed to grow until 80% confluency using chondro-FBS medium (cFBS-M: DMEM high glucose—Gibco USA, Penicillin/Streptomycin/Amphotericin 1% v/v—Gibco USA, Insulin/transferrin/selenium 1% v/v-Thermofisher USA, Dexamethasone 0.1 μM-Sigma-Aldrich USA, L-prolin 40 μg/ml-Sigma-Aldrich USA, Fetal Bovine Serum 10% v/v-Gibco USA).

hACs were gently detached adding 5 ml of TryPLE (Gibco USA) on T75 flask and maintaining at 37° C. for 10 minutes. Cells were recovered through centrifugation at 1200 rpm for 7 minutes (rotor radius=247 mm), then, a pooling of hACs from three donors was performed. The cellular pellet was resuspended using a 10% w/v solution of GelMA (Cellink, USA) in 0.25% w/v lithium phenyl-2,4,6-trimethylbenzoyl-phosphinate (LAP) (Cellink, USA)/PBS solution, avoiding the formation of air bubble during the whole process. Finally, the cell suspension was dispensed into a silicon mold, and a UV-mediated crosslinking was induced irradiating scaffolds with an UV lamp equipped with led at 395 nm (LEPRO, USA) for 3 minutes. Scaffolds were carefully removed from the silicone molds an put in not-treated 24 well plate. Hence, each scaffold was provided with 1 ml of cFBS-M, and incubated at 37° C., 5% CO₂ for 24 hours. At his point, the cFBS-M was switched into chondro complete medium (cCM: cFBS-M+TGFβ-3 10 ng/ml, Ascorbic Acid 50 μg/ml, NO FBS).

### Example 6: Evaluation of hACs Viability Cultured Within a Crosslinked Gelatin-Based Scaffold

Cells or cell-laden scaffolds were washed twice in PBS samples to remove any culture medium residual, then stained using a LIVE/DEAD assay (Abcam, UK). This kit contained two fluorescent dyes, calcein AM and ethidium homodimer-1 (Ethd-1) that selectively stain living (green) and dead (red) cells respectively. Samples were stained using 4 μM calcein and 2 μM Ethd-1 (final concentration) in PBS for 30 minutes at 37° C. and 5% CO₂. After staining, samples were rinsed twice in PBS to remove dye excess and imaged using an inverted epifluorescence microscope EVOS M5000 (Thermofisher, USA).

### Comments to the Results

The experiments reported above confirm that the scaffolds of the invention, doped with magnetic microparticles, are responsive to external cyclic magnetic fields. In fact, the overall diameter of the hydrogel evaluated during the MRI stimulation, equal to 19.69 mm, (FIGS. 2a and 2b) is lower compared to the diameter, equal to 23.83 mm, of the same scaffold measured immediately before the magnetic stimulation (FIG. 3a). Such data suggest that the scaffold of the invention reacts to magnetic field with a contraction of 28.6%. Remarkably, such a deformation is reversible, as the scaffold showed a complete recovery of its initial dimension immediately after the deformation (FIG. 3b), reaching a diameter of 24.72 mm upon relaxation.

The same material enriched with BaTiO₃ as piezoelectric filler showed a responsiveness to a mechanical compression within the same range of deformation. In particular, the experiments show that the material of the invention reacts to compression regimens performed using a strain-control set-up, producing an electric effect in terms of generation of a voltage difference, that is capable to stimulate and promote a pro-regenerative response in the adjoining tissues. Without being bound to any theory, the pro-regenerative signals are delivered in situ by the cells loaded in the material itself, allowing to perform personalized treatments for the patients.

1. A biocompatible composition for use in implants in an area of the body subjected to surgery for the treatment of pathologies characterized from chondral defects, comprising:
a) a matrix comprising a biocompatible polymer that is intrinsically piezoelectric, or a biocompatible polymer non-intrinsically piezoelectric loaded with an inorganic or organic filler imparting piezoelectric properties to the non-intrinsically piezoelectric polymer;
b) between 1×10⁵-1×10⁹ nanoparticles/ml of a magnetic material; and
c) between 1×10⁵ and 1×10⁸ cells chosen from the group consisting of mesenchymal stem cells, adult chondrocytes, and chondrocyte progenitors per milliliter of composition.

2. A biocompatible composition according to claim 1, wherein said intrinsically piezoelectric biocompatible polymer is selected in the group consisting of polyvinylidene difluoride, poly(vinylidene fluoride-trifluorethylene), poly(L-lactic acid), poly(3-hydroxybutyrate-co-3-hydroxyvalerate), cellulose, collagen, polyacrylonitrile, nylon-11, and mixtures thereof.

3. A biocompatible composition according to claim 2, wherein said intrinsically piezoelectric biocompatible polymer is dissolved or suspended in a solvent selected among water, PBS at pH 7.4, culture media, tetrahydrofuran, diethyl ether, dimethylformamide, exafluoroisopropanol, methyl ethyl ketone, and mixtures thereof.

4. A biocompatible composition according to claim 1, wherein the amount of intrinsically piezoelectric biocompatible polymer is between 2 and 30% w/v.

5. A biocompatible composition according to claim 1, wherein said non-intrinsically piezoelectric biocompatible polymer is of natural origin.

6. A biocompatible composition according to claim 5, wherein said non-intrinsically piezoelectric biocompatible polymer is an animal gelatin.

7. A biocompatible composition according to claim 5. wherein the non-intrinsically piezoelectric biocompatible polymer is a hydrogel incorporating water, PBS or culture media.

8. A biocompatible composition according to claim 7, wherein the amount of polymer ranges between about 2 and 30% w/v of the overall composition.

9. A biocompatible composition according to claim 5, wherein said non-intrinsically piezoelectric biocompatible polymer is loaded with micro-or nanoparticles of an inorganic filler having piezoelectric properties.

10. A biocompatible composition according to claim 9, wherein said inorganic filler is selected among barium titanate, lead zirconate titanate, zinc stannate, lithium niobate, sodium niobate and mixtures thereof.

11. A biocompatible composition according to claim 9, wherein said nanoparticles have a diameter or maximum axis between 1 nm and 100 nm, and wherein said microparticles have a diameter or maximum axis between 101 nm and 100 μm.

12. A biocompatible composition according to claim 9, wherein the nanoparticles of inorganic filler are present in the composition in an amount between 4 and 40% w/v.

13. A biocompatible composition according to claim 1, wherein said magnetic material in the form of nanoparticles is selected among a magnetic metal, a magnetic metal oxide, a magnetic metal alloy and mixtures thereof.

14. A biocompatible composition according to claim 1, wherein said magnetic metal is selected among iron, nickel and cobalt, said magnetic metal oxide is an oxide or mixed-oxide of iron, nickel and cobalt, and said magnetic metal alloy is selected among steel, an alloy of the family “AlNiCo”, an alloy of the family “TiCoNiAl”, and an alloy based on rare-earth elements.

15. A biocompatible composition according to claim 1, wherein said magnetic metal oxide is selected between ferric oxide and magnetite, and said alloy based on rare-earth elements is selected between a samarium-cobalt alloy and a neodymium-iron-boron alloy.

16. A biocompatible composition according to claim 13, wherein said magnetic material is present in the composition in an amount between 1×10⁶ and 1×10⁹ nanoparticles/ml.

17. A biocompatible composition according to claim 1, comprising between 1×10⁵ and 1×10⁸ cells chosen from the group consisting of mesenchymal stem cells, chondrocyte progenitors, or adult chondrocytes per milliliter.

18. A biocompatible composition according to claim 17, comprising between 2×10⁶ and 2×10⁷ cells chosen from the group consisting of mesenchymal stem cells, chondrocyte progenitors, or adult chondrocytes per milliliter.

19. A biocompatible composition according to claim 1, further comprising one or more additional components selected among antioxidants, preservatives, and bioactive agents.

20. A biocompatible composition according to claim 19, wherein said bioactive agents are selected among growth factors, drugs, antibiotics, antivirals, enzymes, vitamins, and mixtures thereof.

21. A method for the treatment of an articular disease in a subject in need thereof, said method comprising the step of using the biocompatible composition according to claim 1.

22. The method for the treatment of an articular disease according to claim 21, wherein said subject in need thereof is suffering from a chondral defect.

23. The method for the treatment of an articular disease according to claim 21, wherein said subject in need thereof is suffering from osteoarthritis.
